(19) United States
(12) Patent Application Publication
(10) Pub. No.: US 2013/0084633 A1
(43) Pub. Date: Apr. 4, 2013

(54) PETRI DISHES

(71) Applicants: In Hee SHIN, Gwangju (KR); Hyun Seo Kang, Gwangju (KR); Young Sun Kim, Daejeon (KR); Kwon-Seob Lim, Gwangju (KR); Hyoung Jun Park, Gwangju (KR); Young Soon Heo, Gwangju (KR)

(72) Inventors: In Hee SHIN, Gwangju (KR); Hyun Seo Kang, Gwangju (KR); Young Sun Kim, Daejeon (KR); Kwon-Seob Lim, Gwangju (KR); Hyoung Jun Park, Gwangju (KR); Young Soon Heo, Gwangju (KR)

(73) Assignee: ELECTRONICS AND TELECOMMUNICATIONS RESEARCH INSTITUTE, Daejeon (KR)

(21) Appl. No.: 13/558,959

(22) Filed: Jul. 26, 2012

(30) Foreign Application Priority Data
Sep. 30, 2011 (KR) 10-2011-0099867

(51) Int. Cl. C12M 1/22

(52) U.S. Cl. 435/305.1

(57) ABSTRACT

Provided is a Petri dish, which improves or maximizes productivity. The Petri dish includes a dish including a bottom and a side wall, and accommodating a culture solution, and a cover covering an upper portion of the dish. The bottom of the dish includes a support region having a first thickness, and a measurement region having a second thickness smaller than the first thickness.

### CROSS-REFERENCE TO RELATED APPLICATIONS

This U.S. non-provisional patent application claims priority under 35 U.S.C. §119 of Korean Patent Application No. 10-2011-0099867, filed on Sep. 30, 2011, the entire contents of which are hereby incorporated by reference.

### BACKGROUND OF THE INVENTION

The present invention disclosed herein relates to a Petri dish, and more particularly, to a Petri dish for living cells, which is appropriate for real time high magnification imaging.

Various research efforts are underway all over the world to analyze movement of living cells and medication reaction mechanisms thereof. To this end, various technologies for living cell imaging tools are being developed. However, technologies of imaging living cells in real time are mainly concentrated on the control of external conditions such as temperature and carbon dioxide for growing living cells, and the development of living cell imaging tools are neglected. Living cells may be cultured in a Petri dish. Such Petri dishes may store a culture solution. In addition, Petri dishes may be moved in and out of a culture medium for living cells. Living cells in a Petri dish may be observed using a microscope. Such microscopes may be classified into upright microscopes and inverted microscopes according to measuring directions of an object lens.

An object lens of upright microscopes may be disposed above a Petri dish to magnify a culture solution. The object lens may be immersed in the culture solution to highly magnify the culture solution. In this case, the object lens may be damaged by the culture solution.

An object lens of inverted microscopes may be disposed under a Petri dish to magnify a culture solution. In this case, magnifying power of the object lens may be determined according to a bottom thickness of the Petri dish. Most of Petri dishes have a bottom thickness of about 1 mm or greater to protect a culture solution from the outside.

Accordingly, when an object lens of an inverted microscope is used to magnify a culture solution in a typical Petri dish, the magnifying power thereof is limited to under 40 magnifications.

### SUMMARY OF THE INVENTION

The present invention provides a Petri dish appropriate for real time high magnification measurement of an inverted microscope.

The present invention also provides a Petri dish, which improves or maximizes productivity.

Embodiments of the inventive concept provide Petri dishes including: a dish including a bottom and a side wall, and accommodating a culture solution; and a cover covering an upper portion of the dish, wherein the bottom of the dish includes a support region having a first thickness, and a measurement region having a second thickness smaller than the first thickness.

In some embodiments, the bottom of the dish may include an inner bottom surface contacting the culture solution, and an outer bottom surface disposed under the inner bottom surface.

In other embodiments, the inner bottom surface may include a first flat surface connected to the side wall, and a second flat surface, and a level of the second flat surface from the outer bottom surface may be lower than that of the first flat surface.

In still other embodiments, the outer bottom surface may include a third flat surface.

In even other embodiments, the second thickness may range from the second flat surface to the third flat surface.

In yet other embodiments, the first thickness may range from the first flat surface to the third flat surface.

In further embodiments, the outer bottom surface may include a fourth flat surface that is disposed under the second flat surface and that is higher than the third flat surface.

In still further embodiments, the fourth flat surface may have an area that is equal to or greater than that of the second flat surface.

In even further embodiments, the second thickness may range from the second flat surface to the fourth flat surface.

In yet further embodiments, the second thickness may be about 0.2 mm or smaller.

In much further embodiments, the dish may further comprise a handle protruded along an outer circumferential surface of the side wall.

In still other embodiments, the handle supports the cover.

### BRIEF DESCRIPTION OF THE DRAWINGS

The accompanying drawings are included to provide a further understanding of the present invention, and are incorporated in and constitute a part of this specification. The drawings illustrate exemplary embodiments of the inventive concept and, together with the description, serve to explain principles of the present invention. In the drawings:

FIG. 1 is a plan view illustrating a Petri dish according to an embodiment of the inventive concept;

FIG. 2 is an exploded cross-sectional view taken along line I-I′ of FIG. 1;

FIG. 3 is a cross-sectional view illustrating the Petri dish of FIG. 1 and an object lens of a microscope;

FIG. 4 is a plan view illustrating a Petri dish according to another embodiment of the inventive concept;

FIG. 5 is an exploded cross-sectional view taken along line II-II′ of FIG. 4; and

FIG. 6 is a cross-sectional view illustrating the Petri dish of FIG. 4 and an object lens.

### DETAILED DESCRIPTION OF PREFERRED EMBODIMENTS

Preferred embodiments of the inventive concept will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

In this specification, it will also be understood that when another component is referred to as being ‘on’ one component, it can be directly on the one component, or an intervening third component may also be present. Also, in the figures, the dimensions of components are exaggerated for clarity of illustration. Like reference numerals refer to like elements throughout

The embodiment in the detailed description will be described with cross-sectional views and/or plan views as ideal exemplary views of the present invention. In the figures, the dimensions of layers and regions are exaggerated for clarity of illustration. Accordingly, shapes of the exemplary views may be modified according to manufacturing techniques and/or allowable tolerances. Thus, areas exemplified in the drawings have general properties, and are used to illustrate a specific shape of a device region. Accordingly, this should not be construed as limited to the scope of the present invention. Embodiments described and exemplified herein include complementary embodiments thereof.

In the following description, the technical terms are used only for explaining exemplary embodiments while not limiting the present invention. The terms of a singular form may include plural forms unless referred to the contrary. The meaning of ‘comprises’ and/or ‘comprising’ does not exclude other components besides a mentioned component.

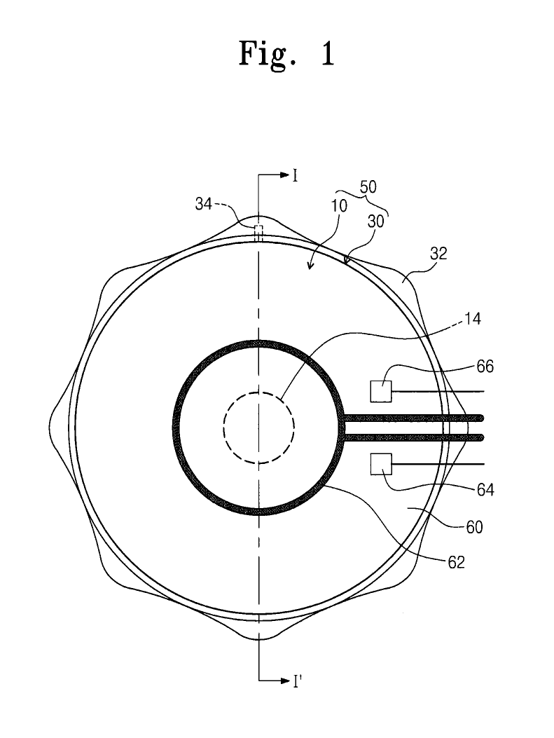

FIG. 1 is a plan view illustrating a Petri dish according to an embodiment of the inventive concept.

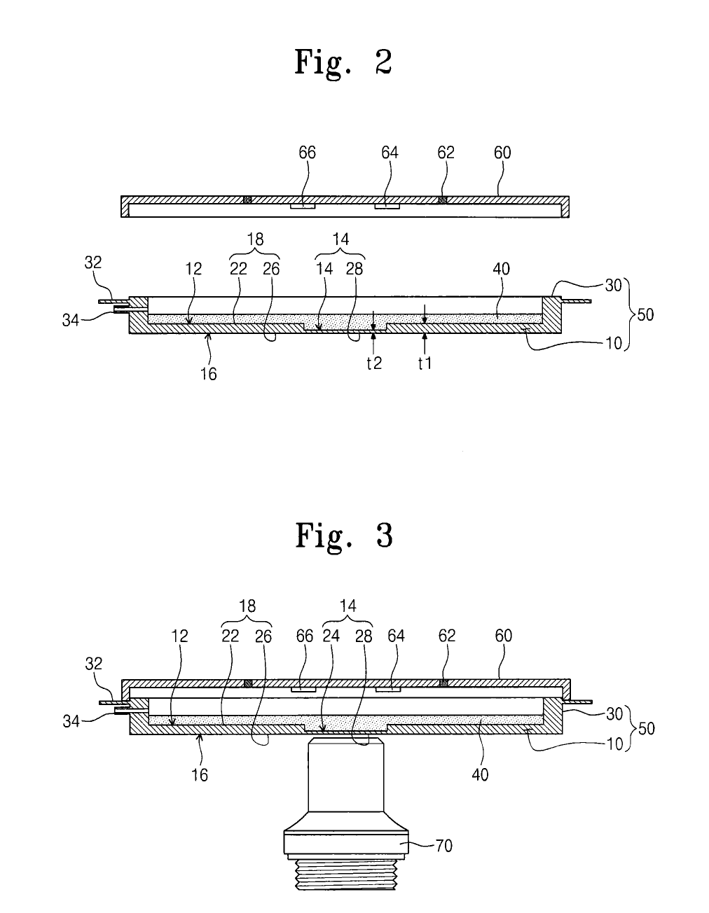

FIG. 2 is an exploded cross-sectional view taken along line I-I′ of FIG. 1. FIG. 3 is a cross-sectional view illustrating the Petri dish of FIG. 1 and an object lens of a microscope.

Referring to FIGS. 1 to 3, a Petri dish 50 according to the embodiment of the inventive concept may include a support region 18 having a first thickness t1, and a measurement region 14 having a second thickness t2 smaller than the first thickness t1. A culture solution 40 may be highly magnified about 40 times or more through the measurement region 14 by an object lens 70. The second thickness t2 may be about 0.2 mm or smaller.

Thus, the Petri dish 50 makes a high magnification measurement of an object lens possible.

The Petri dish 50 may be formed of a transparent plastic material, a polymer material, or a glass material. The Petri dish 50 may include a bottom 10 and a side wall 30 surrounding the edge of the bottom 10. The side wall 30 may surround the edge of the bottom 10, and support a cover 60. The side wall 30 may include: a handle 32 protruding out of the Petri dish 50; and a gas injection inlet 34 for injecting gas such as carbon dioxide into the Petri dish 50. The handle 32 may include protrusions that are repeatedly arrayed along the outer circumferential surface of the side wall 30. The handle 32 may support the cover 60. The gas injection inlet 34 may be higher than the level of the culture solution 40 accommodated in the Petri dish 50. The gas injection inlet 34 may be closed by a cover (not shown) to prevent the culture solution 40 from being contaminated, or discharged.

The bottom 10 may include an inner bottom surface 12 and an outer bottom surface 16. The inner bottom surface 12 may contact the culture solution 40. The outer bottom surface 16 may include a third flat surface 26. The inner bottom surface 12 may include a first flat surface 22 and a second flat surface 24 lower than the first flat surface 22. The first flat surface 22 may be disposed in an edge portion of the bottom 10 near the side wall 30.

The first thickness t1 may range from the first flat surface 22 to the third flat surface 26. For example, the first thickness t1 may be about 1 mm or greater. The second flat surface 24 may be disposed at the center of the bottom 10.

The second thickness t2 may range from the second flat surface 24 to the third flat surface 26. The second thickness t2 may be about 0.2 mm or smaller. The measurement region 14 may have a circular shape. Although not shown, the measurement region 14 may have a triangular, tetragonal, or polygonal shape.

The object lens 70 of an inversed microscope may move close to the third flat surface 26 of the outer bottom surface 16. The object lens 70 may have a focal length of about 1 mm or smaller in order to magnify the culture solution 40 about 40 times or more. The culture solution 40 positioned on the second flat surface 24 may be highly magnified through the measurement region 14 by the object lens 70.

Thus, the Petri dish 50 may make it possible for the object lens 70 to highly magnify and measure the culture solution 40 within the Petri dish 50.

The Petri dish 50 may be formed through economical plastic injection molding or extrusion. The first flat surface 22 and the second flat surface 24 are disposed at different levels from the outer bottom surface 16.

The first flat surface 22 and the second flat surface 24 provide a stepped structure to the bottom 10 without fabrication difficulty. The first flat surface 22 may be connected to the second flat surface 24 through a slope or a vertical surface. That is, the support region 18 and the measurement region 14 have different thicknesses in the bottom 10. The Petri dish 50 including the bottom 10 with a constant thickness difference may be economically formed through injection molding or extrusion. Accordingly, productivity of forming the Petri dish 50 can be improved or maximized.

The cover 60 may cover the upper portion of the Petri dish 50. The cover 60 may include a heater line 62, a temperature sensor 64, and a carbon dioxide sensor 66. The heater line 62 may be heated by voltage from an external power source. The heater line 62 may heat the inside of the Petri dish 50 and the cover 60. The temperature sensor 64 may sense temperature of the cover 60. The temperature sensor 64 may include a thermocouple device. The carbon dioxide sensor 66 may sense carbon dioxide floating within the Petri dish 50 and the cover 60.

The heater line 62 and the temperature sensor 64 may be connected to a temperature adjuster (not shown).

The temperature adjuster may monitor inner temperature of the Petri dish 50. The temperature adjuster may receive a temperature sensing signal from the temperature sensor 64, and control voltage applied from the power source to the heater line 62. The carbon dioxide sensor 66 may include a non-dispersive infrared (NDIR) sensor and a chemical gas sensor.

The cover 60 may be coupled to the Petri dish 50 to protect the culture solution 40. The cover 60 coupled to the Petri dish 50 may provide a space for a culturing circumstance of the culture solution 40, such as temperature and air. The Petri dish 50 and the cover 60 may be economically formed through injection molding or extrusion.

Accordingly, the productivity of forming the Petri dish 50 can be improved or maximized.

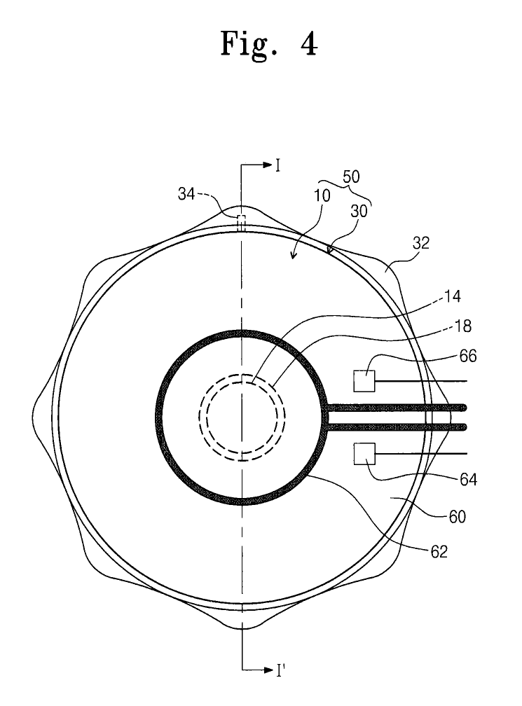

FIG. 4 is a plan view illustrating a Petri dish according to another embodiment of the inventive concept.

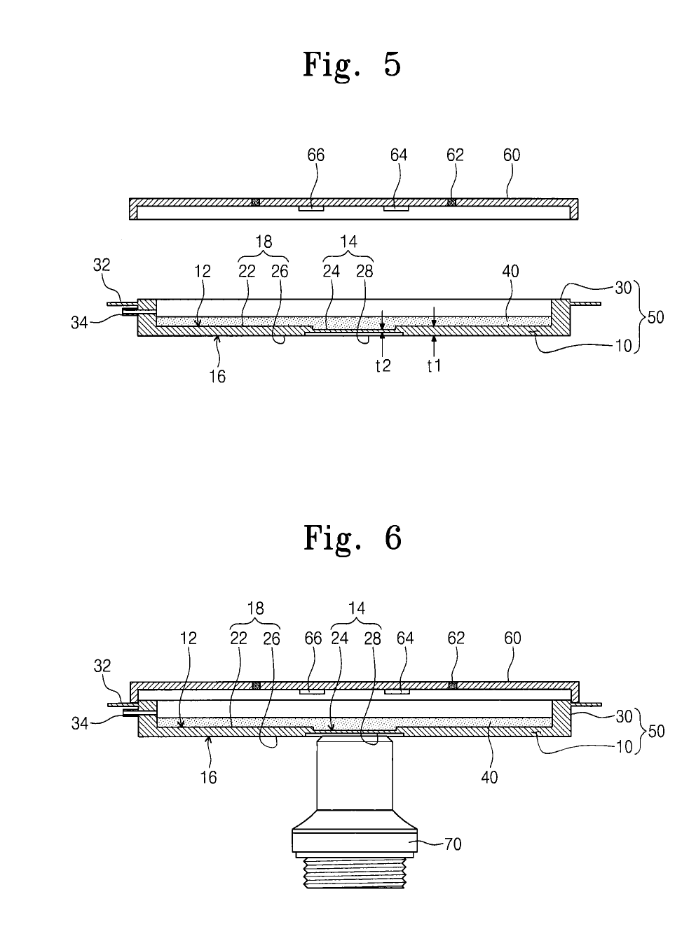

FIG. 5 is an exploded cross-sectional view taken along line II-II′ of FIG. 4. FIG. 6 is a cross-sectional view illustrating the Petri dish of FIG. 4 and an object lens.

Referring to FIGS. 4 to 6, a Petri dish 50 according to the current embodiment may include a measurement region 14 having a second thickness t2 ranging from a second flat surface 24, lower than a first flat surface 22, to a fourth flat surface 28 higher than a third flat surface 26. A support region 18 may have a first thickness t1 ranging from the first flat surface 22 to the third flat surface 26. The first thickness t1 may be about 1 mm or greater. The second thickness t2 may be about 0.2 mm or smaller. An object lens 70 of an inversed microscope may move close to or support the fourth flat surface 28 of the measurement region 14. A culture solution 40 may be highly magnified about 40 times or more through the measurement region 14 by the object lens 70.

Thus, the Petri dish 50 makes a high magnification measurement of the object lens 70 possible.

An inner bottom surface 12 of the Petri dish 50 may include the first flat surface 22 connected to a side wall 30, and the second flat surface 24 lower than the first flat surface 22. The first flat surface 22 may be connected to the second flat surface 24 through a slope or a vertical surface. An outer bottom surface 16 of the Petri dish 50 may include the third flat surface 26 connected to the side wall 30, and the fourth flat surface 28 higher than the third flat surface 26. The third flat surface 26 may be connected to the fourth flat surface 28 through a slope or a vertical surface. The fourth flat surface 28 may have an area equal to or greater than that of the second flat surface 24.

A bottom 10 of the Petri dish 50 may include the support region 18 having the first thickness t1, and the measurement region 14 having the second thickness t2. The inner bottom surface 12 and the outer bottom surface 16 may include the first to fourth flat surfaces 22, 24, 26, and 28. The bottom 10 may include the first to fourth flat surfaces 22, 24, 26, and 28, and slopes or vertical surfaces therebetween. The Petri dish 50 may be economically formed through injection molding or extrusion.

Accordingly, productivity of forming the Petri dish 50 can be improved or maximized.

According to the embodiments of the inventive concept, the bottom of a Petri dish may include a support region having a first thickness, and a measurement region having a second thickness smaller than the first thickness. A culture solution in the dish can be highly magnified through the measurement region by an object lens of a microscope. The support region and the measurement region may include first to fourth flat surfaces. The bottom of the dish may include the first to fourth flat surfaces, and slopes or vertical surfaces connecting the first to fourth flat surfaces. The Petri dish may be economically formed through injection molding or extrusion.

Accordingly, productivity of forming the Petri dish can be improved or maximized.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A Petri dish comprising:
a dish having a bottom and a side wall, and accommodating a culture solution; and
a cover covering an upper portion of the dish,
wherein the bottom of the dish comprises a support region having a first thickness, and a measurement region having a second thickness smaller than the first thickness.

2. The Petri dish of claim 1, wherein the bottom of the dish comprises an inner bottom surface contacting the culture solution, and an outer bottom surface disposed under the inner bottom surface.

3. The Petri dish of claim 2, wherein the inner bottom surface comprises a first flat surface connected to the side wall, and a second flat surface,
and a level of the second flat surface from the outer bottom surface is lower than that of the first flat surface.

4. The Petri dish of claim 3, wherein the outer bottom surface comprises a third flat surface.

5. The Petri dish of claim 4, wherein the second thickness ranges from the second flat surface to the third flat surface.

6. The Petri dish of claim 5, wherein the first thickness ranges from the first flat surface to the third flat surface.

7. The Petri dish of claim 4, wherein the outer bottom surface comprises a fourth flat surface that is disposed under the second flat surface and that is higher than the third flat surface.

8. The Petri dish of claim 7, wherein the fourth flat surface has an area that is equal to or greater than that of the second flat surface.

9. The Petri dish of claim 7, wherein the second thickness ranges from the second flat surface to the fourth flat surface.

10. The Petri dish of claim 1, wherein the second thickness is about 0.2 mm or smaller.

11. The Petri dish of claim 1, wherein the dish further comprising a handle protruded along an outer circumferential surface of the side wall.

12. The Petri dish of claim 10, wherein the handle supports the cover.
